(19) United States
(12) Patent Application Publication
(10) Pub. No.: US 2005/0048129 A1
(43) Pub. Date: Mar. 3, 2005

(54) Methods and compositions to aid breast enhancement

(76) Inventor: Kristin Monday, Incline Village, NV (US)

Correspondence Address: FISH & RICHARDSON, PC, 12390 EL CAMINO REAL, SAN DIEGO, CA, 92130-2081, US

(21) Appl. No.: 10/651,528

(22) Filed: Aug. 29, 2003

(51) Int. Cl. A61K 35/80; A61K 35/55; A61K 35/78

(52) U.S. Cl. 424/565; 424/195.17; 424/727; 424/750; 424/745; 424/757; 424/773; 424/764; 424/778; 514/567

(57) ABSTRACT

The invention provides a composition, comprising a glandular agent; a kelp derivative; and a pituitary extract. The compositions are useful to increase breast size in a subject.

### TECHNICAL FIELD

This invention relates to methods and compositions useful for increasing breast size.

### BACKGROUND

Breast development begins at puberty, when the body receives chemical signals from the pituitary gland. These signals orchestrate changes throughout the body including the stimulation of breast development. Research into breast development suggests that breast tissue growth occurs in response to hormonal signals including estrogen, progesterone, prolactin, human and growth hormone. Lack of these compounds can lead to underdevelopment of breasts during puberty. In addition, body metabolism and fat and water retention can play a role in breast development.

### SUMMARY

The invention provides a composition, comprising a glandular agent; a kelp derivative; and a pituitary extract. In one aspect of the invention the composition further comprises an agent selected from the group consisting of Fenugreek, Saw Palmetto, Mexican Wild Yam, Fennel, Dong Quai Damiana, Blessed thistle, L-Tyrosine, Mothers Wort, Black Cohosh, Oat Grass, and Hops flower. The glandular agent is obtained from a non-human species such as a bovine species. In one aspect of the invention the glandular is bovine ovary.

The invention also provides a composition of the invention comprising a pharmaceutically acceptable excipient/carrier.

In another aspect of the invention a method of increasing breast size in a subject is provided. The method includes administering the composition of the invention to the subject in an amount sufficient to increase breast size.

The details of one or more embodiments of the invention are set forth in the description herein. Other features, objects, and advantages of the invention will be apparent from the description and from the claims.

### DETAILED DESCRIPTION

The interest in non-surgical breast enhancement has increased over the past decade as risks associated with implants (e.g., silicon and saline based implants) have become further characterized. In addition, studies have shown that women with small breast size have lower self-esteem and poor body image. The invention uses non-surgical compositions and methods to increase breast size in subjects. The compositions and methods of the invention stimulate a subject's metabolism and hormone balance to a direction characteristic of early breast development.

The methods and compositions of the invention provide a therapy that stimulates fatty accumulation in the breast thereby increasing their size. Larger breasts are comprised mainly of fatty tissue held together by connective tissue. All women have approximately an equal number of mammary glands. During puberty one's body releases growth hormones that cause fat to accumulate. The compositions of the invention stimulate the body to release these same developmental hormones that in turn cause an accumulation of fatty tissue and thus increased breast size.

The invention provides a composition comprising a glandular (e.g., bovine ovary), pituitary extract, and a kelp-derivative. The compositions of the invention are typically taken orally three-times per day, although dosing will depend upon the weight, size, and sex of the subject. Such dosing will be readily apparent by those of skill in the art and/or can be determined empirically.

The compositions of the invention comprise from about 75-90% by weight a glandular agent, 5-24% by weight a pituitary extract, and 1-5% by weight a kelp derivative. For example, in one aspect of the invention the composition comprises 75% by weight bovine ovary (as the glandular agent), 24% by weight pituitary extract, and 1% a kelp derivative. Other agents can be added to the composition including Fenugreek, Saw Palmetto, Mexican Wild Yam, Fennel, Dong Quai Damiana, Blessed thistle, L-Tyrosine, Mothers Wort, Black Cohosh, Oat Grass, and Hops flower. Should other agents be added, the percentage can be modified accordingly, so that the sum of the percentage by weight of all ingredients is 100%.

Kelp (fucus vesiculosus) is an excellent source of minerals from the sea, including iodine, which is important for the thyroid to function properly. Studies regarding diets including kelp have determined a link to a lower breast cancer rate, and a healthier hormonal balance. Kelp is a source of vitamins A, B1, B2, C, D and E, plus amino acids. It contains algin, which will absorb toxins from the digestive tract. Bladderwrack kelp is one of the richest natural sources of approximately 30 trace elements and major minerals. It regulates the thyroid function and may be helpful in reducing obesity where it is associated with thyroid trouble. Bladderwrack kelp is also a metabolic stimulant. This is important to keep tissue in the breasts, as well as elsewhere in the body, healthy. Typical parts of a kelp plant that can be used in the methods and compositions of the invention include the dried thallus and the fresh thallus of the bladderwrack. Some thallus ends look grainy and it is here that the reproductive organs are found. The fructifications consisting of 3 cm long ovoid receptacles are found in the tips of these thalli and are either cordate or ovately flattened with grainy bladders. The bladderwrack plant is often over 1 m long, olive green when fresh, black brown when dry. The stem of the thallus is flat, repeatedly bifurcated and has a midrib along the whole length. Beside this midrib there are often scattered pores and numerous air-filled bladders. The plant is found on the North Sea coast, the western Baltic coast, and on the Atlantic and Pacific coasts. Bladderwrack consists of the dried thallus of Fucus vesiculosus, of Ascophyllum nodosum Le Jolis, or of both species, as well as preparations of same. Other names associated with Bladderwrack include Seawrack, Kelpware, Black-tang, Bladder Fucus, Cutweed, Fucus, Quercus marina, Sea-Wrack, and Kelp-Ware.

Sources of kelp are known in the art. For example, the kelp is provided by picking fresh kelp and allowing it to dry to a stage where it can be finely ground or otherwise comminuted. Alternatively, it is from previously dried kelp, whole, or previously ground to a desired size. The dried kelp (or part thereof) particles are dispersed or dissolved in an aqueous media such as the aqueous medium described herein below. The ground particle size useful in the compositions of the invention is about 0.1-20 μm, or 0.2-10 μm, but is typically about 0.2-5 μm. Alternatively, an extract of kelp may also be prepared by steam distillation, expression (hard pressing), or maceration. A tinture extract can be diluted as appropriate to obtain the desired concentration and/or therapeutic effect. Other methods of preparing kelp can be found in, “The Homoeopathic Pharmacopoeia,” Official Compendium, Jul. 1, 1992, Pharmacopoeia Convention of the American Institute of Homeopathy (Publishers), Falls Church, Virginia, incorporated herein by reference.

The kelp product present in the compositions of the invention are useful for stimulating thyroid function and metabolism. The thyroid function increases energy by regulating metabolism that boosts thermogenesis (i.e., burns calories). The micronutrients in kelp enhance stamina and help to balance hormones. A kelp derivative useful in the invention includes tinctures and leaf or plant parts or particles derived from a kelp plant.

Glandular tissue, as used herein, includes a gland tissue or organ that supplies biologically active hormones, hormone precursors, enzymes, vitamins, minerals, soluble proteins, and natural lipid factors. Such glandulars offer a diversity of hormones and hormone related substances in a natural, non-toxic quantity for therapeutic, rejuvenative and preventive health care. Glandular supplements have been used in medicine for thousands of years. A gland is defined as any organ that secretes substances into the bloodstream. The main concept behind glandular therapy is that the ingestion of glandular tissue (usually bovine in origin) provides the hormones, enzymes and other biologically active substances that are normally secreted by the corresponding gland in the human body. In other words, “like matches like” and supplementation of adrenal gland, for example, will help support the function of the adrenal glands in the body. While glandular therapy usually involved the use of fresh, whole glands, modern glandular therapy typically uses concentrated extracts of the glands.

Glandulars for use in the methods and compositions of the invention may be obtained from any source including bovine, porcine, and other mammalian tissue sources. For example, bovine glandulars are a readily available source of such glandulars as the ovary gland. Bovine ovary compositions are capable of stimulating the biological pathways similar to the human ovary. Thus, bovine ovary is a rich source of hormones and factors that stimulate the estrogen levels and other growth factor hormones in the body. This stimulation of hormonal pathways simulates hormonal levels that are seen during puberty and pregnancy of women, times of development when most women see growth in their breasts. Such glandulars, including bovine ovary, are typically lyophilized (freeze-dried in the raw state) to provide the highest levels of biological activity and maintain the naturally occurring nutrients including vitamins, minerals, enzymes, nucleoproteins and lipoproteins.

The pituitary gland, which is often referred to as the “master gland,” regulates the release of most of the body's hormones. The pituitary gland controls the release of thyroid, adrenal, growth and sex hormones. The hypothalamus regulates the release of hormones from the pituitary gland. Many of the hormones released by the pituitary are involved in the control of puberty including GnRH (gonadotropin releasing hormone); estrogen, a female sex hormone, which is responsible for breast development; leutinizing hormone (LH), which stimulates gonadal development and regulation; follicle-stimulating hormone (FS), which stimulates gonadal development and regulation; samototropin, growth hormone (HGH) which effects most tissues of the body; and prolactin which stimulates breast tissue and gonads. Prolactin is likely the biggest stimulator of breast development. Prolactin is a protein hormone principally effecting breast development and milk production. Prolactin is known to be a principle stimulator of original development of breast tissue and its further hyperplasia during pregnancy. Prolactin, along with other pituitary hormones that stimulate gonadal tissue (e.g., estrogen and progesterone), growth hormone, and the like stimulate proliferation and branching of the ducts in the female breast.

Ingestion of such pituitary extracts provided by the compositions of the invention provide the hormones, enzymes and other biologically active substances that are normally secreted by the pituitary in the human body. These active substances are provided in addition (i.e., as a supplement) to those in the body and in some cases, where the body no longer produces such factor, renews those activities associated with release of the factors in vivo.

The compositions of the invention may be formulated using a safe and effective amount of the three main ingredients discussed above to provide one or more of the beneficial effects of the invention described herein. One or more of the optional ingredients described herein may be further included in the compositions and formulations of the invention. The compositions of the invention may be formulated with a pharmaceutically acceptable carrier.

Typically the composition comprising the three ingredients described herein are formulated in orally acceptable dosages including, but not limited to, capsules, tablets, lozenges, troches, hard candies, powders, sprays, gels, elixirs, syrups, and suspensions or solutions.

The pharmaceutically acceptable carrier may include, but is not limited to: (a) carbohydrates including sweeteners; typically including fructose, sucrose, sugar, dextrose, starch, lactose, maltose, maltodextrins, corn syrup solids, and honey solids; (b) sugar alcohols including mannitol, sorbitol, xylitol, and (c) various relatively insoluble excipients including dicalcium phosphate, calcium sulfate, calcium carbonate, microcrystalline cellulose and other pharmaceutical tableting ingredients.

Lozenges, tablets and troches of the invention are essentially the same, but may differ in shape, size and manufacturing technique.

In the case of tablets, for oral use, the pharmaceutically acceptable carrier may further include lactose and corn starch. Lubricating agents may also be added to the tablets, including, for example, magnesium stearate, sodium lauryl sulfate and talc. Tablets may also contain excipients such as sodium citrate, calcium carbonate and calcium phosphate. Disintegrants such as starch, alginic acid and complex silicates, may also be employed. Tablets may also include binding agents such as polyvinylpyrrolidone, gelatin, PEG-8000 and gum acacia.

In the case of lozenge's for oral use, the common pharmaceutically acceptable carrier may further include a binder such as PEG-8000. Typically lozenges are made in a 0.1 to 15 grams size to allow a suitable dissolution rate for lozenges.

To directly make lozenges the active ingredients are added to PEG-8000 processed fructose; or add the active ingredient to crystalline fructose. Add saccharin if desired, flavors as desired, glidants such as silica gel as needed, and lubricants such as magnesium stearate as needed. The mixture should be kept dry and tableted soon after mixing. The ingredients are mixed and directly compressed into lozenges using conventional pharmaceutical mixing and tableting equipment. The compressive force is preferably sufficient to produce maximum hardness throughout the lozenges to preserve the dissolution rate and maximize the efficacy of lozenges.

Tablets and troches can be manufactured using procedures similar to that described above with minor changes in the optional ingredients.

Alternatively, the compositions of the invention may be formulated in liquid form, such as syrups or sprays with a solvent or dispersant such as water, or other liquids in a pharmaceutically acceptable carrier for repeated delivery of the compositions of the invention to oral and oropharyngeal mucous membranes over a sustained period of time.

In one aspect, the invention provides an aqueous medium comprising a glandular agent, a kelp derivative, and a pituitary extract. As mentioned above the composition comprising the aqueous medium may also include other active agents such as Fenugreek, Saw Palmetto, Mexican Wild Yam, Fennel, Dong Quai Damiana, Blessed thistle, L-Tyrosine, Mothers Wort, Black Cohosh, Oat Grass, and Hops flower.

Acceptable aqueous vehicles for use in the compositions and methods of the invention include, for example, any liquid solution that is capable of dissolving, or generating a suspension of a glandular agent, a kelp derivative, and a pituitary extract and which is not toxic to the particular subject receiving the composition. Examples of acceptable aqueous vehicles include, without limitation, saline, water, and acetic acid.

General methods of formulating an aqueous medium can be found in, for example, “Remington's Pharmaceutical Sciences.” For example, formulations for delivery of the compositions of the invention may contain aqueous solutions comprising, polyoxyethylene-9-lauryl ether, glycocholate, and deoxycholate, or may be oily solutions.

The aqueous medium typically comprises water and typically includes other materials such as surfactants, vitamins and vitamin derivatives, antihistamines, wetting agents, preservatives, moisturizers, emulsifiers, odorants, and the like, present in conventional concentrations. Those skilled in the art will have no difficulty in determining suitable materials and concentrations for their known functions. In one aspect a general formulation may comprise: a glandular agent, a kelp derivative, a pituitary extract, and water and optionally flavoring is then the balance of the composition. Where a saline solution is desirable the aqueous medium may comprise a small amount of dissolved sodium chloride in the aqueous medium. The salt concentration may be in the range of 0.1-2.0% and will typically be on the order of about 0.65% to 0.9%. The NaCl concentration may vary, but is preferably at a normal hysiological NaCl concentration.

The compositions of the invention may be formulated in capsule form with or without diluents. For capsules, useful diluents include lactose and dried corn starch. When suspensions are employed, emulsifying and/or suspending agents may be employed in the suspensions. In addition, solid compositions including one or more of the ingredients of the lozenges described above may be employed in soft and hard gelatin capsules.

Other materials, which may optionally be included in the formulations of the invention include inositol, other B-complex vitamins, and anti-inflammatories. Also, ingredients such as sweeteners, flavorants, coloring agents, dyes, preservatives, emulsifying agents, suspending agents, melting agents, excipients, and solvents or diluents such as water, ethanol, propylene glycol, glycerin and various combinations thereof, may be included in the compositions of the invention.

The optional sweeteners which may be used in the formulation of the invention include, but are not limited to, saccharin, aspartame, cyclamates, acesulfame K, neohesperidin dihydrochalcone, other super sweeteners, and mixtures thereof, which may be added to the carrier in amounts sufficiently low so as not to chemically interact with the main ingredients of the composition.

The optional flavorants which may be used in the compositions and formulations of the invention include, but are not limited to, peppermint, peppermint-menthol, eucalyptol wintergreen, licorice, clove, cinnamon, spearmint, cherry, lemon, orange lime, menthol and various combinations thereof.

The three main ingredients described above which may be derived from kelp, pituitary, and glandular tissue, make up from about 0.5-90% by weight of the total composition of the formulation.

The compositions/formulations may be administered 1-6 times per day, as needed, or more commonly, 1-3 times per day, as needed. As discussed above, the compositions of the invention may be administered to a person in any orally acceptable dosage form including, but not limited to tablets, capsules, lozenges, troches, hard candies, powders, sprays, gels, elixirs, syrups, and suspensions or solutions.

The invention relates to a method of administering to a subject an amount of the composition of the invention, which is effective to provide an increase in breast size during a course of treatment.

The effective amount of the composition will vary depending on such factors as the subject being treated, the particular mode of administration, the activity of the particular active ingredients employed, the age, bodyweight, general health, sex and diet of the subject, time of administration, rate of excretion, the particular combination of ingredients employed, and the total content of the main ingredients of the composition. It is within the skill of the person of ordinary skill in the art to account for these factors.

As noted above, the particular route of administration can influence the effective amount and duration of treatment with the composition of the invention as well as the frequency of administration. For example, orally administered agents may require higher concentrations to deliver an effective amount to a target area or tissue than administration to a mucus membrane.

A number of embodiments of the invention have been described. Nevertheless, it will be understood that various modifications may be made without departing from the spirit and scope of the invention. Accordingly, other embodiments are within the scope of the following claims.

1. A composition, comprising
a glandular agent;
a kelp derivative; and
a pituitary extract.

2. The composition of claim 1, further comprising an agent selected from the group consisting of Fenugreek, Saw Palmetto, Mexican Wild Yam, Fennel, Dong Quai Damiana, Blessed thistle, L-Tyrosine, Mothers Wort, Black Cohosh, Oat Grass, and Hops flower.

3. The composition of claim 1, wherein the glandular agent is obtained from a non-human species.

4. The composition of claim 1, wherein the glandular agent is a bovine glandular.

5. The composition of claim 4, wherein the bovine glandular is a bovine ovary.

6. The composition of claim 1, wherein kelp derivative is a dried thallus and/or a fresh thallus of a kelp plant.

7. The composition of claim 1, wherein the kelp derivative is obtained from bladderwrack.

8. The composition of claim 1, wherein the pituitary extract is from a non-human animals species.

9. The composition of claim 1, further comprising a pharmaceutically acceptable carrier.

10. A method of increasing breast size in a subject, comprising
administering a composition comprising
a glandular agent;
a kelp derivative; and
a pituitary extract,
to the subject in an amount sufficient to increase breast size.

11. The method of claim 10, wherein the composition further comprises an agent selected from the group consisting of Fenugreek, Saw Palmetto, Mexican Wild Yam, Fennel, Dong Quai. Damiana, Blessed thistle, L-Tyrosine, Mothers Wort, Black Cohosh, Oat Grass, and Hops flower.

12. The method of claim 10, wherein the glandular agent is obtained from a non-human species.

13. The method of claim 10, wherein the glandular agent is a bovine glandular.

14. The method of claim 13, wherein the bovine glandular is a bovine ovary.

15. The method of claim 10, wherein kelp derivative is a dried thallus and/or a fresh thallus of a kelp plant.

16. The method of claim 10, wherein the kelp derivative is obtained from bladderwrack.

17. The method of claim 10, wherein the pituitary extract is from a non-human animals species.

18. The method of claim 10, further comprising a pharmaceutically acceptable carrier.
